(19) United States
(12) Patent Application Publication
(10) Pub. No.: US 2006/0236433 A1
(43) Pub. Date: Oct. 19, 2006

(54) Nicotiana hybrids and plant varieties for use in production of pharmaceuticals

(75) Inventors: David Zaitlin, Lexington, KY (US); Richard Mundell, Lexington, KY (US)

Correspondence Address: BUCHANAN, INGERSOLL & ROONEY PC, POST OFFICE BOX 1404, ALEXANDRIA, VA, 22313-1404, US

(73) Assignee: University of Kentucky, Lexington, KY (US)

(21) Appl. No.: 11/390,343

(22) Filed: Mar. 28, 2006

Related U.S. Application Data
(60) Provisional application No. 60/665,350, filed on Mar. 28, 2005.

(51) Int. Cl. A01H 5/00; C12N 9/00; C12N 15/82; C07K 16/18; C07K 14/415; C07K 1/00

(52) U.S. Cl. 800/288; 800/317.3; 530/370; 530/388.1; 435/183

(57) ABSTRACT

The present invention relates to a method of producing a non-food crop Nicotiana plant that is optimized for producing plant-manufactured biologicals. These can be industrial or research enzymes or proteins, as well as pharmaceutical or therapeutic proteins such as vaccines, antigens, enzymes, antibodies, etc. that can be isolated and purified and administered to a subject. The invention also discloses sterile interspecific Nicotiana hybrids with high biomass produced by the method of the invention, and the seeds, tissue cultures, methods of selecting these plants and regenerating these plants.

### CROSS REFERENCE TO RELATED APPLICATION

The application claims priority under 35 U.S.C. §119 to U.S. Provisional Application No. 60/665,350 filed Mar. 28, 2005, the entire content of which is hereby incorporated herein by reference.

### FIELD OF THE INVENTION

The present invention relates to non-food/feed hybrid Nicotiana plant varieties and hybrids as host plants for the purpose of producing plant-made pharmaceuticals (PMPs) and proteins including, but not limited to therapeutic immunoglobulins, immunoglobulin derivatives, medical enzymes, research enzymes, industrial enzymes and vaccines.

### BACKGROUND OF THE INVENTION

With the steadily increasing demand for biopharmaceuticals, the production of pharmaceuticals in plants is expected to become an important field. Traditional crop systems already in place are not effective for pharmaceutical production, as these crop species are selectively bred for traditional applications, such as food production. Further, the use of food crops for production of pharmaceuticals is discouraged due to identity preservation and genetic containment issues. Thus, it is desirable to use non-food plants, such as Nicotiana, for the production of plant-made pharmaceuticals (PMPs) and other protein-based biologicals. There is a need in the art for plant vehicles that are compatible with common gene expression systems, but also display effective identity preservation and genetic containment sufficient to satisfy federal regulatory requirements.

### SUMMARY OF THE INVENTION

Therefore, notwithstanding what has previously been reported in the literature, there exists a need for improved non-food/feed plant varieties and hybrid plants for producing plant-made proteins and other biologicals. Additionally, methods of making and using sterile Nicotiana hybrids and varieties for plant-made pharmaceutical (PMP) production will greatly aid PMP technology and acceptance, and will simplify governmental regulatory oversight.

An aspect contemplates a method of producing an interspecific F₁ Nicotiana hybrid for producing a plant-made pharmaceutical. Preferably the pharmaceutical is a protein, peptide or polypeptide. The method comprises the steps of: a) choosing a transgenic Nicotiana maternal line expressing a protein of interest, wherein said transgenic Nicotiana maternal line further has attributes optimal for producing the plant-made protein; b) choosing a Nicotiana paternal line of a different species than said transgenic Nicotiana maternal line, wherein said Nicotiana paternal line further has additional attributes optimal for producing the plant-made protein; and c) crossing said transgenic Nicotiana maternal line with said Nicotiana paternal line to produce the interspecific F₁ Nicotiana hybrid; and d) wherein said interspecific F₁ Nicotiana hybrid is an interspecific diploid hybrid which is not chromosome doubled, and wherein said interspecific F₁ Nicotiana hybrid is both male sterile and female sterile. Another aspect contemplates that the Nicotiana paternal line can further express one or more additional proteins of interest. The maternal line can also contain and express more than one nucleic acid, which produces a peptide or polypeptide of interest. Proteins of interest include, but are not limited to immunoglobulins (e.g., antibodies, antibody fragments and derivatives), vaccines, antigens, enzymes, and enzyme subunits. The maternal and paternal Nicotiana lines can express the same nucleic acid or different nucleic acids. Therefore, for example, they can express the same antibody, different antibodies, or an antibody and enzyme.

Another aspect of the invention contemplates a method of producing an interspecific F₁ Nicotiana hybrid for producing a plant-made pharmaceutical such as a protein comprising: a) choosing a Nicotiana maternal line that may or may not be transgenic, wherein said Nicotiana maternal line has attributes optimal for producing the plant-made protein; b) choosing a Nicotiana paternal line of a different species than said transgenic Nicotiana maternal line, wherein said Nicotiana paternal line further has additional attributes optimal for producing the plant-made protein, and wherein said Nicotiana paternal line is transgenic, and further expresses a protein of interest; and c) crossing said Nicotiana maternal line with said Nicotiana paternal line to produce the interspecific F₁ Nicotiana hybrid; and d) wherein said interspecific F₁ Nicotiana hybrid is an interspecific diploid hybrid which is not chromosome doubled, and wherein said interspecific F₁ Nicotiana hybrid is both male sterile and female sterile.

In yet another embodiment, the methods contemplated above can have the paternal and/or the maternal lines expressing one more of the following characteristics

(i) distinctive plant morphology for identity preservation;

(ii) compatibility with efficient growing and mechanical harvesting practices;

(iii) rapid plant growth;

(iv) high plant biomass production;

(v) high yield of biomass from repeated harvesting of leaf and stem tissue;

(vi) reduced susceptibility to a Nicotiana disease; and/or

(vii) reduced susceptibility to a Nicotiana pest;

in any combination of one or more.

The Nicotiana paternal line can be any Nicotiana plant. More preferably it is a Nicotiana sp. discussed herein. Yet more preferably, the Nicotiana paternal line is N. benthamiana, N. glauca, N. glutinosa, N. quadrivalvis, N. otophora, or N. sylvestris.

The Nicotiana maternal line can be any Nicotiana plant. More preferably it is a Nicotiana sp. discussed herein. Yet more preferably, the Nicotiana maternal line is a selected cultivar of N. tabacum.

Yet another contemplated aspect is an interspecific F₁ Nicotiana hybrid produced by any of the methods discussed above and herein. The interspecific F₁ Nicotiana hybrid can display one or more of the following characteristics: (i) is unable to transmit foreign genes to related plant species or native plants, due to sterility; (ii) is easily harvested by a mechanized process; and (iii) is readily distinguishable by its external appearance from other crop plants, thus ensuring that it may be identity-preserved. A tissue culture of the hybrids produced by any of these methods is also contemplated as is any plant produced from the tissue cultures.

Another aspect of the invention contemplates a plant made pharmaceutical or protein biological produced by any interspecific F₁ Nicotiana hybrid produced. Preferably the plant made pharmaceutical is a peptide or polypeptide/protein. Preferable proteins include but are not limited to an industrial enzyme, a research enzyme, a vaccine, an immunoglobulin, or an immunoglobulin derivative.

Yet a further aspect of the invention contemplates a method of producing interspecific F₁ Nicotiana hybrids for producing a plant-made pharmaceutical comprising: a) choosing a Nicotiana maternal line which expresses a compound of interest, wherein said Nicotiana maternal line further has attributes optimal for producing plant-made proteins; b) choosing a Nicotiana paternal line of different species than said Nicotiana maternal line, wherein said Nicotiana paternal line further has additional attributes optimal for producing plant-made proteins; and c) crossing said Nicotiana maternal line with said Nicotiana paternal line to produce the interspecific F₁ Nicotiana hybrid; and d) wherein said interspecific F₁ Nicotiana hybrid is an interspecific diploid hybrid which is not chromosome doubled, and wherein said F₁ Nicotiana hybrid is male sterile and female sterile. Another aspect contemplates that the compound of interest is a peptide, protein or polypeptide. Additionally, it is a further aspect that the Nicotiana maternal line is obtained using an alternative gene-expression system. It is further contemplated that the Nicotiana paternal line is obtained using an alternative gene-expression system. Another aspect of the invention contemplates that the method described above uses a maternal and/or paternal line which has one or more of the following attributes:

(i) distinctive plant morphology for identity preservation;

(ii) compatibility with efficient growing and harvesting practices;

(iii) rapid plant growth;

(iv) high plant biomass production;

(v) high yield of biomass from repeated harvesting of leaf and stem tissue;

(vi) reduced susceptibility to a Nicotiana disease; and

(vii) reduced susceptibility to a Nicotiana pest.

The paternal line can be any of the Nicotiana plants lines known, more preferably any of those discussed herein, and more preferably wherein the line is a selected cultivar or variety of N. benthamiana, N. glauca, N. glutinosa, N. quadrivalvis, N. otophora, or N. sylvestris. Likewise the maternal Nicotiana plant can be any Nicotiana plant discussed herein, but is preferably a selected cultivar of N. tabacum. Also contemplated is an interspecific F₁ Nicotiana hybrid produced by this method, a tissue culture of the hybrid, and a plant produced from the tissue culture.

An interspecific F₁ Nicotiana hybrid as described above, wherein said hybrid further has at least one of the following characteristics: (i) is unable to transmit foreign genes to related plant species or native plants, due to <99% sterility; (ii) is easily harvested by mechanized processes; and (iii) is readily distinguishable by its external appearance from other crop plants, thus ensuring that it may be identity-preserved. Another aspect contemplates the protein, polypeptide, or peptide produced by said plant. The protein can be but is not limited to an industrial enzyme, a research enzyme, a vaccine, an immunoglobulin, or an immunoglobulin derivative.

### DETAILED DESCRIPTION OF THE INVENTION

It has been newly found that interspecific crosses between common or cultivated tobacco (N. tabacum) and other species of Nicotiana produce vigorous, male and female sterile hybrid plants that are morphologically distinct from conventional tobacco, thus achieving identity preservation. These F₁ hybrid plants can be grown in a manner similar to cultivated tobacco, although preferably at a higher plant density than is typically achieved with cultivated tobacco. These plants can be cultivated using existing infrastructure (e.g., float beds, planting and harvesting machinery, fertilizers, herbicides, insecticides, and fungicides), yet can be mechanically harvested three or four times during the growing season. We have also found that several Nicotiana species, other than N. tabacum, can be used as maternal parents for sterile interspecific hybrids that are suitable for plant-made protein production.

For production of pharmaceutical, medical, or industrial proteins or polypeptides, the maternal Nicotiana species or cultivar is transformed with a gene of interest, and homozygous transformants preferably with high-level protein expression are selected and crossed with other selected Nicotiana species. It is the resulting F₁ hybrid offspring that are transplanted and grown in the field for plant-made protein production. The maternal species can be a cultivar of N. tabacum or another species of Nicotiana. In another aspect, the paternal species of the F₁ hybrid can also be transformed with a gene encoding a protein or polypeptide of interest.

1. Definitions and Abbreviations

The following definitions and methods are provided to better define the present invention and to guide those of ordinary skill in the art in the practice of the present invention. It must be noted that as used herein and in the appended claims, the singular forms “a” or “an” or “the” include plural referents unless the context clearly dictates otherwise. Thus, for example, reference to “a protein” includes a plurality of such proteins and reference to “the antibody” includes reference to one or more antibodies and equivalents thereof known to those skilled in the art, and so forth.

Unless defined otherwise, all technical and scientific terms used herein have the same meaning as commonly understood to one of ordinary skill in the art to which this invention belongs.

1.1 Definitions

By “plant” as used herein include any Nicotiana plant. The Nicotiana plants are preferably a non-food, non-feed crop plant that is cultivated for the purpose other than as one of food for humans or animals, such as domesticated animals. By “Nicotiana” is meant the genus in the family Solanaceae of approximately 70 species of plants native to the Americas, Australia, and Africa. Nicotiana is meant to preferably include varieties of N. tabacum (common tobacco), as well as species such as N. glauca, N. benthamiana, N. glutinosa, N. otophora and N. sylvestris. Other Nicotiana species (and their varieties) are contemplated by the term Nicotiana, but are not limited to: N. acaulis, N. acuminata, N. africana, N. alata, N. amplexicaulis, N. arentsii, N. attenuata, N. azambujae, N. benavidesii, N. bigelovii, N. bonariensis, N. cavicola, N. clevelandii, N. cordifolia, N. corymbosa, N. debneyi, N. eastii, N. excelsior, N. exigua, N. forgetiana, N. fragrans, N. goodspeedii, N. gossei, N. heterantha, N. ingulba, N. kawakamii, N. knightiana, N. Iangsdorffii, N. linearis, N. longibracteata, N. longiflora, N. maritima, N. megalosiphon, N. miersii, N. nesophila, N. noctiflora, N. nudicaulis, N. obtusifolia, N. occidentalis, N. paa, N. paniculata, N. pauciflora, N. petunioides, N. plumbaginifolia, N. quadrivalvis, N. raimondii, N. repanda, N. rosulata, N. rotundifolia, N. rustica, N. Xsanderae, N. setchellii, N. simulans, N. solanifolia, N. spegazzinii, N. stocktonii, N. suaveolens, N. thyrsiflora, N. tomentosa, N. tomentosiformis, N. umbratica, N. undulata, N. velutina, and N. wigandioides.

By “N. tabacum” is meant to include any cultivars and varieties that are members of this species. Preferred cultivars include ‘Turkish Samsun’, SN2108, VA509, KY171, Havana 38, K326, TN90, TI 1122 (Venezuela), TI 1127 (Venezuela), TI 162 (Japan), and TI 179 (Columbia) and other high-yielding cultivars, and those with desirable attributes for PMP production. The TI 162 (Japan) variety is characterized by large plants that remain standing in the field after frosts have killed most or all other varieties of tobacco; the TI 162 variety is also late flowering. The TI 179 (Columbia) variety is characterized by tall plants with large, upright leaves, which sustain very little insect damage.

By “biomass” is meant the total fresh weight in kilograms of plant tissue which is harvested above the ground. The terms “fresh weight” and “biomass” are used interchangeably herein. Preferred plants have a high biomass of at least about 80,000 kg/hectare, more preferably of at least about 90,000 kg/hectare, and most preferably a plant with a biomass of up to about 120,000 kg/hectare from multiple harvests.

By “host” is meant a plant cell, tissue or entire plant organism capable of supporting replication of a replicable vector containing a nucleic acid of interest (such as a DNA that encodes a polypeptide or gene that encodes a protein) that encodes a peptide, polypeptide or protein. The host should also be capable of being infected by a virus containing the viral vector or plant viral nucleic acid.

By “infection” is meant the ability of a virus to transfer its nucleic acid to a host plant or introduce viral nucleic acid into a host plant, wherein the viral nucleic acid is replicated, viral proteins are synthesized, and new viral particles assembled. In this context, the terms “transmissible” and “infective” are used interchangeably herein.

By “polypeptide” and “protein” as used herein, unless otherwise indicated, are meant to be those moieties encoded by a nucleic acid of interest.

By “phenotypic trait” is meant an observable morphological or biochemical property resulting in the present context, from the expression of genes in the hybrid state. Preferred phenotypic traits of the contemplated hybrid plants include one or more of the following traits: greater biomass than the original parent plants; good identity preservation to prevent co-mingling with similar commercial crops; inability to transmit foreign genes to related crop species; is a non-food crop so that the transgenic PMP varieties cannot enter the human or animal food chain; be a plant that is readily transformed and regenerated by existing methodologies; be compatible with viral vector transformation systems (e.g., tobacco mosaic virus—TMV—or other plant viral-based vectors); be capable of producing the PMPs or protein biologicals at levels greater than about 1% total soluble proteins (TSP); have acceptable chemical profiles (e.g., endogenous levels of alkaloids and phenolics); be tolerant of environmental stresses such as cold or drought; express resistance and tolerance to insects and diseases; grow rapidly; and be easily harvested using existing machinery. For example, easily harvestable plants include those that grow well in higher density than standard tobacco culture (32,000 plants/acre versus 8,000 plants/acre), and those that will also produce secondary shoots (regrowth) following harvest, which lend themselves to multiple mechanized harvestings.

As used herein, “plant tissue” is any tissue of a plant in planting or in culture. This term is intended to include a whole plant, plant cell(s), plant organ, protoplast, cell culture, or any group of plant cells organized into a structural and functional unit.

As used herein, “recombinant plant virus” is a plant virus containing the recombinant plant viral nucleic acid and additional sequences of interest. Examples of such recombinant plant viruses include, but are not limited to, vectors based on tobacco mosaic virus (TMV), tobacco rattle virus (TRV), potato virus X (PVX), tomato bushy stunt virus (TBSV), sonchus yellow net virus (SYNV) and barley stripe mosaic virus (BSMV).

As used herein, a “vector” is a self-replicating moiety, typically a nucleic acid molecule comprising preferably the nucleic acid of interest that is operably linked to the vector molecule, and is capable of transferring the nucleic acid of interest between plant cells.

As used herein, “backcrossing” is a process in which a breeder repeatedly crosses hybrid progeny back to one of the parents, for example, a first generation hybrid F₁ with one of the parental genotypes of the F₁ hybrid. If this process is repeated for multiple generations with the same (recurrent) parental line, it is known as “recurrent selection.” By “infertile” is meant a plant that produces essentially no viable pollen or egg cells. In this case, interspecific Nicotiana hybrids are infertile (sterile), because the chromosomes of the plants fail to pair properly at meiosis. The interspecific Nicotiana hybrids lack a doubled-chromosomal complement, and thereby produce <1% viable gametes. Such plants will be both male and female sterile and will thus be unable to transmit any genes to other non-food crop plants. By “interspecific hybrid” is meant F₁ hybrid plants formed from the sexual crossing of two different species within a genus (in this case Nicotiana).

By “parent plant” is meant the parent Nicotiana plant used to cross with another Nicotiana plant to obtain the interspecific hybrid Nicotiana plant.

By “readily regenerable” as used in context with plant regeneration is meant plant material that can be induced to form vegetative shoots and roots in tissue culture by one skilled in this practice using established protocols for tobacco. The resulting rooted vegetative shoots can then be grown in soil to produce fertile adult plants.

By “readily transformable” as used in context with plant transformation is meant plant material into which foreign DNA (genes) can be introduced by one skilled in this practice using established protocols for tobacco; either through the use of transforming strains of Agrobacterium and the appropriate DNA vectors, or via particle bombardment or other suitable methods.

By “immunoglobulin” is meant to include polyclonal or monoclonal antibodies, different immunoglobulin classes (e.g., IgM, IgA, IgG) and subclasses (IgG₁ to IgG₄), and fragments thereof (e.g., Fab, F(ab′)₂, and scFv).

1.2 Abbreviations

The following abbreviations are used in the application:
BSMV barley stripe mosaic virus
F₁ First filial generation hybrid between two (2) true-breeding varieties of a species (intraspecific hybrid) or between two compatible species within a genus (interspecific hybrid)
GM genetically modified
ORF open reading frame
PMP plant made pharmaceutical
PVX potato virus X
sp. species
SYNV sonchus yellow net virus
TBSV tomato bush stunt virus
TMV tobacco mosaic virus
TRF tobacco rattle virus
   2.0 Expression of Proteins in Nicotiana

Methods for expressing genes in plants to produce proteins are known. See for example R. Walden, GENETIC TRANSFORMATION IN PLANTS (Open University Press, 1988); Elizabeth E. Hood et al., PLANTS AS FACTORIES FOR PROTEIN PRODUCTION (Kluwer Academic Publishers, 2002); Charles Cunningham et al., RECOMBINANT PROTEINS FROM PLANTS: PRODUCTION AND ISOLATION OF CLINICALLY USEFUL COMPOUNDS (Humana Press, 1997); Karen Harper et al., RECOMBINANT ANTIBODIES: APPLICATIONS IN PLANT SCIENCE AND PLANT PATHOLOGY (CRC Press, 1999); and in U.S. Pat. Nos. 5,316,931; 5,589,367; 5,811,653; and 5,866,785; all of which are incorporated herein in their entirety for all purposes.

Efforts directed towards enhancing plant-made pharmaceuticals (PMP) production usually focus on increasing protein yields by optimizing gene expression in transgenic plants, targeting gene products to subcellular compartments, stable transformation of the chloroplast genome or by development of efficient recombinant viral vectors. Little or no attention is given to the development of plant varieties designed for PMP production in a field setting. In order for molecular farming to become a viable part of the agriculture sector, real-world agronomic considerations must be addressed.

Nicotiana is a genus of about 70 species of tobacco native to the Americas, Australia and Africa, some of which are listed supra. There are several species of Nicotiana that contribute valuable characteristics, such as disease resistance, insect resistance, tolerance to cold and drought, rapid growth, desirable chemical profile, and identity preservation. It is newly determined that several undomesticated species of Nicotiana can be crossed with cultivated common tobacco (N. tabacum) to produce sterile, high-yielding hybrids, which can be designed to express traits preferable for production of PMPs.

2.1 Expression of PMP Expressing Nucleic Acids in Nicotiana

N. tabacum (cultivated tobacco) is a highly diverse species comprising several distinct types, including burley, dark-cured, flue-cured, cigar wrapper, cigar filler, primitive, and oriental (Turkish). N. tabacum has a long history as both a plant grown for the production of tobacco (non-edible crop plant) and a research model.

Common laboratory cultivars of N. tabacum, such as ‘Petite Havana’, ‘Xanthi’, ‘Wisconsin 38’, ‘Samsun’, and ‘Turkish Samsun’ are readily transformed and regenerated, but may perform poorly in a field setting. Cultivars are available of N. tabacum, which produce greater than about 50,000 kg of green biomass/acre/year under intensive cultivation conditions with multiple harvests (e.g., KY171, K326, NC297, Havana 38). High biomass hybrids are preferred as plants with high biomass produce more PMP for harvest, whereas lower biomass hybrids have less. Recombinant proteins are produced in the leaves but also can be found in the other parts of the non-food crop plant such as Nicotiana.

The present invention is directed to interspecific hybrids between N. tabacum and other Nicotiana species, such as but not limited to N. sylvestris, N. glauca, N. otophora, N. glutinosa, N. quadrivalvis, and N. benthamiana, which are very useful in molecular farming applications and PMP production. To this end, the hybrids of the present invention have the advantage of being produced through straightforward crosses, are both >99% male and female sterile, and are easily distinguished visually from conventional tobacco (identity preservation). These hybrids grow very well in Kentucky. Finding alternative uses for tobacco is of interest to tobacco farmers and the agricultural community of the state, as well as to other states, which are known for growing tobacco.

The successful production of pharmaceuticals and protein biologicals in plants requires a gene of interest and an appropriate method for introducing the gene of interest into a plant host, as well as a transformable and regenerable plant host. Methods for selecting for the presence of the gene and to regenerate the transformed plants and to separate and purify the desired plant product from the other plant constituents are required.

As opposed to hybrids known in the art, the present invention does not select for one parental species based on properties relating to viral activity and high-level protein expression from infection with a recombinant viral vector. Nor does the present invention select for a fertile, doubled-chromosome hybrid. Instead, the hybrids of the present invention expressing foreign proteins (i.e., PMPs) are both male- and female-sterile (<1% viable gametes) interspecific hybrids. Thus, the hybrids of the present invention have the advantage in that they cannot transmit any genes to tobacco, or any other plants, growing nearby. Furthermore, the sterility of the hybrids results in a high level of genetic containment, satisfying federal regulatory requirements.

The hybrids of the present invention can be planted and grown in a manner similar to conventional tobacco using existing fertilizers, machinery, herbicides and float beds, with the exception that the plants have the capacity of being planted at a higher plant density (about 32,000 versus about 8,000 plants/acre) with subsequent multiple harvestings by machine.

For the production of proteins, the maternal species or cultivar may be transformed with a gene of interest. Homozygous transformants with high-level protein expression are selected and crossed with another Nicotiana species. The resulting F₁ hybrid offspring are grown in the field. In some applications, the paternal parent of the F₁ sterile hybrid(s) could also be similarly transformed with the same or a different gene of interest.

### EXAMPLE 1

### Hybrid Nicotiana of N. tabacum and N. benthamiana

Materials and Methods. Flowers of the selected tobacco (N. tabacum) maternal cultivar(s) were emasculated by removing the pollen-bearing structures (the anthers) just prior to anthesis. This was usually accomplished 1-2 days before the flower bud opened. The expanded corolla tube, which is a structure composed of the fused petals of the flower in Nicotiana, was slit lengthwise to expose the internal structures, and the anthers (which contain the pollen) were removed with small forceps. Care was taken to avoid damage to the stigma and style, the female reproductive structures. Pollen of the paternal line was then applied to the receptive stigma, and the flower was covered with a bag to protect it from adventitious pollen sources. To ensure that the maternal tobacco cultivar would not self-pollinate, any open flowers were removed, and several flowers were emasculated and bagged without being pollinated to serve as negative controls. All emasculated flowers were marked with a paper tag giving the date of emasculation, the date of pollination (if different), and the identity of the pollen source used. Each pollination event was given a unique numerical identifier, which was recorded, along with the identities of the parental lines, in a hybrid database. Plants were then maintained in isolation from one another, under biosafety conditions if warranted, for a period of 30 days. When the seed capsules turned brown and dehisced matured, the seed was collected into labeled seed envelopes and stored under strict containment conditions required for transgenic seed.

Results. Seed set on the N. tabacum maternal parent was good, and the seeds had >80% viability. The resulting F₁ hybrid grew as a small shrub of less than 1 meter in height and flowered 35-40 days after setting. This F₁ hybrid grew fast (i.e., regrew rapidly after harvesting) and could be harvested again in 3-4 weeks. This F₁ hybrid also showed promise in disease resistance tests.

### EXAMPLE 2

### Hybrid Nicotiana of N. excelsior and N. quadrivalvis

Materials and Methods. Procedures for emasculation, pollination and seed collection were as described in Example 1, with the exception that the maternal parent was N. excelsior and the paternal parent was N. quadrivalvis in this example.

Results. This F₁ hybrid obtained grew as a small shrub of less than 1 meter in height and flowered early. This F₁ hybrid grew fast and regenerated rapidly after harvesting. This F₁ hybrid is expected to have good potential for use with modified plant viral vectors.

### EXAMPLE 3

Hybrid Nicotiana of N. tabacum and N. glauca

Materials and Methods. Procedures for emasculation, pollination and seed collection are as discussed in Example 1, with the exception that the paternal parent was N. glauca in this case. The maternal plant was again N. tabacum.

Results. Seed set on the N. tabacum maternal parent was good (˜1,400 seeds per capsule), and the seeds had >80% viability. The resulting F₁ hybrids grew rapidly and flowered 45-50 days after setting at a height of 1.5-2 meters. This F₁ hybrids regrew rapidly after harvesting and produced more shoots and leaves than the N. tabacum parent. The hybrids could be harvested again in 3-4 weeks.

### EXAMPLE 4

### Hybrid Nicotiana of N. tabacum and N. otophora

Materials and Methods. Procedures for emasculation, pollination and seed collection are as discussed in Example 1, with the exception that the paternal parent was N. otophora in this case.

Results. Seed set on the N. tabacum maternal parent was good, and the seeds had ˜60% viability. The resulting F₁ hybrid grew rapidly and flowered very late, approximately 65-70 days after setting, at a height of >2 meters. This F₁ hybrid regrew rapidly after harvesting and produced more shoots and leaves than the N. tabacum parent. The hybrid could be harvested again in 3-4 weeks.

### EXAMPLE 5

### Hybrid Nicotiana of N. tabacum and N. sylvestris

Materials and Methods. Procedures for emasculation, pollination and seed collection are as discussed in Example 1, with the exception that the paternal parent was N. sylvestris in this case. The maternal plant was again N. tabacum.

Results. Seed set on the N. tabacum maternal parent was very good, and the seeds had >80% viability. The resulting F₁ hybrid grew rapidly and flowered approximately 45-50 days after setting, at a height of ˜1.5 meters. The F₁ hybrids regrew rapidly after harvesting, and produced more shoots and leaves than the N. tabacum parent. The F₁ hybrid could be harvested again in 3-4 weeks. This particular F₁ hybrid did not show good identity preservation (it closely resembles standard tobacco varieties), and showed limited fertility in pollination tests.

### EXAMPLE 6

### Hybrid Nicotiana of N. glauca and N. tabacum

Materials and Methods. Procedures for emasculation, pollination and seed collection are as described in Example 1 above, with the exception that N. glauca was the maternal parent, and N. tabacum was the paternal parent in this example. This is the reciprocal of the interspecific cross of Example 3.

Results. There was no apparent seed-set on the N. glauca maternal parent. The immature seed capsules aborted and dropped off the plant 5-7 days after application of N. tabacum pollen. Thus, no plants of this reciprocal hybrid were grown for evaluation in the field.

Interspecific hybridization is relatively common in higher plants, especially in large diverse genera such as Nicotiana. However, because of the large differences in chromosome number and other, possibly uncharacterized barriers to hybridization or progeny fertility in Nicotiana, it is not readily obvious which species pairs will produce viable hybrid seed and if so, at what level. We have analyzed the results of hundreds of controlled pollinations, which has led us to our present state of knowledge regarding Nicotiana interspecific hybrids suitable for plant-made protein production.

### EXAMPLE 7

### Demonstration of Sterility in Interspecific Nicotiana hybrids between N. tabacum and N. glauca

The following hybrids were prepared using the methods discussed in Example 1. The relative fertility of each plant was assessed and is tabulated below:

TABLE 1

| | No. of | | | Pods | | |
|---|---|---|---|---|---|---|
| Nicotiana | Self | | | with | Seeds | Seeds |
| Hybrid | Flowers | Aborted | Pods | Seeds | Produced | Germinated |
| Hybrid 63 | 156 | 156 | 0 | 0 | 0 | 0 |
| Hybrid 83 | 142 | 142 | 0 | 0 | 0 | 0 |
| Hybrid 94 | 163 | 163 | 0 | 0 | 0 | 0 |
| Hybrid 143 | 134 | 134 | 0 | 0 | 0 | 0 |
| Hybrid 150 | 164 | 164 | 0 | 0 | 0 | 0 |

The hybrid crosses for each are as follows: 63—N. tabacum NC297×N. glauca; 83—N. tabacum VA509×N. glauca; 94—N. tabacum KY171×N. glauca; 143—N. tabacum hybrid (S-9-30×S-9-32) N. glauca; 150—N. tabacum ‘Havana 38’×N. glauca.

The next table documents the results of pollen transfer from the parental lines onto several of the hybrids (attempted backcrossing).

TABLE 2

| | No. of | | Pods | Pods with | Seeds | Seeds |
|---|---|---|---|---|---|---|
| Pollen Transfer | Transfers | Aborted | Formed | Seeds | Produced | Germinated |
| Hyb. 150 × paternal line | 9 | 7 | 2 | 0 | 0 | 0 |
| Hyb. 150 × Maternal line 1 | 8 | 6 | 2 | 0 | 0 | 0 |
| Hyb. 150 × Maternal line 2 | 4 | 4 | 0 | 0 | 0 | 0 |
| Maternal line 1 × Hyb. 150 | 6 | 5 | 1 | 1 | 55* | 44* |
| Maternal line 2 × Hyb. 150 | 5 | 5 | 0 | 0 | 0 | 0 |
| Maternal line 3 × Hyb. 150 | 3 | 0 | 3 | 0 | 0 | 0 |
| Maternal line 4 × Hyb 150 | 1 | 1 | 0 | 0 | 0 | 0 |
| Hyb. 94 × Paternal line | 21 | 15 | 6 | 6 | 56 | 19 |
| Hyb. 94 × Maternal line 1 | 11 | 9 | 2 | 0 | 0 | 0 |
| Hyb. 94 × Maternal line 2 | 4 | 3 | 1 | 0 | 0 | 0 |
| Maternal line 1 × Hyb. 94 | 11 | 11 | 0 | 0 | 0 | 0 |
| Maternal line 2 × Hyb. 94 | 2 | 2 | 0 | 0 | 0 | 0 |
| Maternal line 3 × Hyb. 94 | 2 | 2 | 0 | 0 | 0 | 0 |
| Hyb. 94a × Hyb. 94b | 3 | 3 | 0 | 0 | 0 | 0 |
| Hyb. 143 × Paternal line | 8 | 1 | 7 | 1 | 2 | 1 |
| Hyb. 143 × Maternal line 2 | 4 | 4 | 0 | 0 | 0 | 0 |
| Maternal line 2 × Hyb. 143 | 10 | 5 | 5 | 0 | 0 | 0 |
| Maternal line 3 × Hyb. 143 | 4 | 4 | 0 | 0 | 0 | 0 |
| Maternal line 4 × Hyb. 143 | 1 | 0 | 1 | 0 | 0 | 0 |
| Hyb. 63 × Paternal line | 6 | 5 | 1 | 1 | 47 | 24 |
| Hyb. 83 × Paternal line | 5 | 5 | 0 | 0 | 0 | 0 |
| Hyb. 83 × Maternal line 2 | 3 | 3 | 0 | 0 | 0 | 0 |
| TOTALS | | | | | | |
| Hyb. × Paternal line | 49 | 33 | 16 | 8 | 105 | 44 |
| Hyb. × Maternal line | 34 | 29 | 5 | 0 | 0 | 0 |
| Maternal line × Hyb. | 48 | 38 | 10 | 1 | 55* | 44* |

“Hyb.” Stands for Nicotiana hybrid and the number of the hybrid is the same as discuused for Table 1.
By “maternal line #” is menat any of 4 distinct N. tabcum cultivars used in the F1 hybrids as shown in Table 1.
By “Hyb. 94a and Hyb. 94b” are meant two different individuals of Hybrid 94.
By “*” is meant seeds that were most likely produced as a result of pollen contamination from a viable pollen source (such as a tobacco cultivar) during the pollen transfer.
So far, the seed produced by backcrossing onto these interspecific Nicotiana hybrids have produced weak, slow growing seedlings that are not expected to survive past the seedling stage.

Although the present invention has been described in detail with reference to examples above, it is understood that various modifications can be made without departing from the spirit of the invention, and would be readily known to the skilled artisan. It will further be understood by those skilled in the art that various changes in form and details may be made therein without departing from the spirit and scope of the invention as defined by the appended claims.

All cited patents and publications referred to in this application are herein incorporated by reference in their entirety for all purposes.

We claim:

1. A method of producing an interspecific F₁ Nicotiana hybrid for producing a plant-made protein biological comprising:
a) choosing a transgenic Nicotiana maternal line expressing a protein of interest, wherein said transgenic Nicotiana maternal line further has attributes optimal for producing the plant-made protein;
b) choosing a Nicotiana paternal line of a different species than said transgenic Nicotiana maternal line, wherein said Nicotiana paternal line further has additional attributes optimal for producing the plant-made protein; and
c) crossing said transgenic Nicotiana maternal line with said Nicotiana paternal line to produce the interspecific F₁ Nicotiana hybrid; and
d) wherein said interspecific F₁ Nicotiana hybrid is an interspecific diploid hybrid which is not chromosome doubled, and wherein said interspecific F₁ Nicotiana hybrid is both male sterile and female sterile.

2. The method of claim 1, wherein said Nicotiana paternal line further expresses a second protein of interest.

3. The method of claims 1 and/or 2, wherein the protein of interest is an immunoglobulin, a vaccine, an antigen, an enzyme, or an enzyme subunit.

4. The method of claim 3, wherein the immunoglobulin is an antibody or an antibody fragment.

5. The method of claim 3, wherein the protein of interest of the maternal line and the paternal line is an immunoglobulin and the immunoglobulin is an antibody.

6. The method of claim 2, wherein the protein of interest of the maternal line is also expressed in the Nicotiana paternal line.

7. A method of producing an interspecific F₁ hybrid for producing a plant-made protein comprising:
a) choosing a transgenic Nicotiana maternal line, wherein said transgenic Nicotiana maternal line has attributes optimal for producing the plant-made protein;
b) choosing a Nicotiana paternal line of a different species than said transgenic Nicotiana maternal line, wherein said Nicotiana paternal line further has additional attributes optimal for producing the plant-made protein, and wherein said Nicotiana paternal line further expresses a protein of interest; and
c) crossing said transgenic Nicotiana maternal line with said Nicotiana paternal line to produce the interspecific F₁ Nicotiana hybrid; and
d) wherein said interspecific F₁ Nicotiana hybrid is an interspecific diploid hybrid which is not chromosome doubled, and wherein said interspecific F₁ Nicotiana hybrid is both male sterile and female sterile.

8. The method of either claim 1 or 7, wherein said attributes of the paternal line and/or the maternal line are selected from the group consisting of one or more of the following:
(i) distinctive plant morphology for identity preservation;
(ii) compatibility with efficient growing and harvesting practices;
(iii) rapid plant growth;
(iv) high plant biomass production;
(v) high yield of biomass from repeated harvesting of leaf and stem tissue;
(vi) reduced susceptibility to a Nicotiana disease; and
(vii) reduced susceptibility to a Nicotiana pest.

9. The method of claim 1, wherein said Nicotiana paternal line is N. benthamiana, N. glauca, N. glutinosa, N. quadrivalvis, N. otophora, or N. sylvestris.

10. The method of claim 9, wherein said transgenic Nicotiana maternal line is Nicotiana tabacum.

11. An interspecific F₁ Nicotiana hybrid produced by the method of claim 1.

12. The interspecific F₁ Nicotiana hybrid of claim 11, wherein said hybrid further has one or more of the following characteristics:
(i) is unable to transmit foreign genes to related plant species or native plants, due to sterility;
(ii) is easily harvested by a mechanized process; and
(iii) is readily distinguishable by its external appearance from other crop plants, thus ensuring that it may be identity-preserved.

13. A tissue culture of the interspecific F₁ Nicotiana hybrid of claim 11.

14. A plant produced from the tissue culture of claim 13.

15. A plant made protein produced by an interspecific F₁ Nicotiana hybrid of claim 11.

16. The plant made protein of claim 15, wherein the plant made protein is an industrial enzyme, a research enzyme, a vaccine, an antigen, an immunoglobulin, or an immunoglobulin derivative.

17. A method of producing interspecific F₁ Nicotiana hybrid for producing a plant-made pharmaceutical comprising:
a) choosing a Nicotiana maternal line which expresses a compound of interest, wherein said Nicotiana maternal line further has attributes optimal for producing plant-made proteins;
b) choosing a Nicotiana paternal line of different species than said Nicotiana maternal line, wherein said Nicotiana paternal line further has additional attributes optimal for producing plant-made protein; and
c) crossing said Nicotiana maternal line with said Nicotiana paternal line to produce the interspecific F₁ Nicotiana hybrid; and
d) wherein said interspecific F₁ Nicotiana hybrid is an interspecific diploid hybrid which is not chromosome doubled, and wherein said F₁ Nicotiana hybrid is male sterile and female sterile.

18. The method of claim 17, wherein said Nicotiana maternal line expresses the compound of interest which is a peptide, a protein, or a polypeptide, and wherein said Nicotiana maternal line is obtained using an alternative gene-expression system.

19. The method of claim 17, wherein said Nicotiana paternal line expresses the compound of interest, which is a peptide, a protein, a polypeptide, and wherein said Nicotiana paternal line is obtained using an alternative gene-expression system.

20. The method of any of claims 17, 18, and 19, wherein expression of said compound of interest is obtained by transfection of the interspecific F₁ Nicotiana hybrid with an alternative genetic transfection system.

21. The method of claim 17, wherein said attributes of said Nicotiana paternal line and/or said Nicotiana maternal line are selected from the group consisting of one or more of the following:
(i) distinctive plant morphology for identity preservation;
(ii) compatibility with efficient growing and harvesting practices;
(iii) rapid plant growth;
(iv) high plant biomass production;
(v) high yield of biomass from repeated harvesting of leaf and stem tissue;
(vi) reduced susceptibility to a Nicotiana disease; and/or
(vii) reduced susceptibility to a Nicotiana pest.

22. The method of claim 17, wherein said Nicotiana paternal line is N. benthamiana, N. glauca, N. glutinosa, N. quadrivalvis, N. otophora, or N. sylvestris.

23. The method of claim 22, wherein said Nicotiana maternal line is Nicotiana tabacum.

24. An interspecific F₁ Nicotiana hybrid produced by the method of claim 17.

25. A tissue culture of the interspecific F₁ Nicotiana hybrid of claim 24.

26. A plant produced from the tissue culture of claim 25.

27. The interspecific F₁ Nicotiana hybrid of claim 24, wherein said hybrid further has at least one of the following characteristics:
(i) is unable to transmit foreign genes to related plant species or native plants, due to sterility;
(ii) is easily harvested by mechanized process; and/or
(iii) is readily distinguishable by its external appearance from other crop plants, thus ensuring that it may be identity-preserved.

28. A plant made protein produced by said interspecific F₁ Nicotiana hybrid of claim 27.

29. The plant made protein of claim 28, wherein the protein is an industrial enzyme, a research enzyme, a vaccine, an antigen, an immunoglobulin, or an immunoglobulin derivative.
